SEQUENCE LISTING (1 sequence)

SEQ ID NO: 1 - protein, 10 aa, Homo sapiens
  PEPTIDE (1)..(10)
NYRWRCKNQN

(19) United States
(12) Patent Application Publication
(10) Pub. No.: US 2013/0315828 A1
(43) Pub. Date: Nov. 28, 2013

(54) KIT AND METHOD OF USE OF TARGETING PEPTIDE FOR DIAGNOSIS AND THERAPY OF CANCER

(71) Applicants: Margaret Dah-Tsyr Chang, Taipei City (TW); Shun-Lung Fang, Taipei City (TW); Chien-Jung Chen, Changhua County (TW)

(72) Inventors: Margaret Dah-Tsyr Chang, Taipei City (TW); Shun-Lung Fang, Taipei City (TW); Chien-Jung Chen, Changhua County (TW)

(73) Assignee: NATIONAL TSING HUA UNIVERSITY, HSINCHU (TW)

(21) Appl. No.: 13/478,616

(22) Filed: May 23, 2012

(51) Int. Cl. A61K 38/08; A61P 35/00; A61K 49/00; C07K 7/06; C12N 5/09; C12Q 1/04; B82Y 15/00

(52) U.S. Cl. 424/9.1; 435/375; 514/19.3; 435/34; 530/328; 977/773; 977/927

(57) ABSTRACT

The present invention relates to a method of delivering an agent to a cancer cell, comprising: (a) obtaining a peptide that selectively binds to a cancer cell, wherein the peptide comprises a cancer targeting motif having the amino acid sequence of SEQ ID NO: 1, wherein the peptide is attached or fused to an agent that one desires to target to a cancer cell; and (b) exposing the peptide to a population of cells suspected of containing cancer cells. The present invention also relates to a kit for applying the above method, comprising a peptide that binds to a cancer cell, wherein the peptide comprises a cancer targeting motif having the amino acid sequence of SEQ ID NO: 1.

### FIELD OF THE INVENTION

The present invention relates to a kit and method of delivering an agent to a cancer cell for diagnosis or therapy of cancer.

### BACKGROUND OF THE INVENTION

Glycosaminoglycans (GAGs) are linear polysaccharides composed of repeating disaccharide units that are variously modified along their lengths and structures. Their synthesis is not template-driven. Sulfated GAGs such as heparin/heparan sulfate (HS) and chondroitin sulfate (CS) are synthesized, linked through serine-glycine concensus motifs along the core protein as proteoglycans and located on the plasma membrane and in the extracellular matrix (ECM). HS does not occur as free GAG chains in tissues but it is attached to core proteins of six classes of HS proteoglycans (HSPGs). These include the cell-surface glycosylphosphatidylinositol (GPI) anchored glypican family (glypicans 1-6) and transmembrane syndecan family (syndecans 1-4), perlecan, type XVIII collagen, agrin, and the part-time HS co-receptor proteoglycans betaglycan, neuropilin-1, 2 and CD-44 variants (epican). As one group of the most complex biological polymers, HSPGs are a superfamily of components to provide structural frameworks, mediate cell-cell communication by regulating the gradient formation and signaling activities of growth factors, cytokines and morphogens, and the localization and activity of extracellular enzymes such as matrix metalloproteinases (MMPs). HSPGs also function in growth factor-receptor binding to regulate many biological processes in cell growth, development, differentiation, morphogenesis, tissue homeostasis, matrix-remodeling and migration. Thus, HSPGs are key players in molecular networks driving biological phenomena such as development, inflammation, immune response and cancer.

Eosinophils are granulocytes developing during haematopoiesis in the bone marrow before migrating into blood, they are white blood cells and immune system components responsible for combating multicellular parasites and infections in vertebrates. Eosinophil circulation lasts only 2 to 3 days and the migration into tissues is followed by in situ eosinophilic degranulation and lysis. Tissue eosinophil activation with degranulation takes place with in situ interleukine-5 (IL-5) neosynthesis and extracellular release of constitutive eosinophil granular proteins including eosinophil cationic protein (ECP) and eosinophil derived neurotoxin (EDN). Eosinophil infiltration is detected in histological preparations from various tumor tissues including breast, cervical, colon, and lung. Combination of eosinophil granular proteins and cytokines has demonstrated anti-cancer activities and may mediate apoptotic destruction of tumor cells. Along with mast cells, eosinophils control mechanisms associated with allergy and asthma. ECP and EDN are also known as human ribonuclease 3 (RNase 3) and RNase 2, respectively. ECP is mainly expressed in tissues including liver, spleen, and placenta and has a number of different functional properties including ribonucleolytic, cytotoxic, anti-bacteria, anti-virus, anti-parasite and heparin binding activities. In addition, extracellular deposits of ECP are found in tissues undergoing eosinophilic inflammation associated with tissue damage in the case of bronchial asthma or Crohn's disease. ECP binds cell surface GAGs, especially HS on bronchial epithelial cells and enters the cells by lipid-rafted dependent macropinocytosis. The structure of ECP has been determined and refined to a resolution up to 1.75 Å displaying a folding topology with three c′ helices and five 13 strands. The most interesting feature is as many as 19 surface-oriented arginine residues, conferring a strong basic character to ECP (pI=10.8) and facilitating the interaction between ECP and negatively charged molecules on the cell surface.

HS structurally related to heparin binds a wide range of different growth factors, taking part in various physiological and pathological processes including nutritional metabolism, would healing, cell signaling, morphogenesis, cellular crosstalk. ECP contains a major heparin binding motif, RWRCK, located in the loop 3 region between helix α2 and strand β1 (Fan, T. C., Chang, H. T., Chen, I. W., Wang, H. Y., and Chang, M. D. (2007) A heparan sulfate-facilitated and raft-dependent macropinocytosis of eosinophil cationic protein, Traffic 8, 1778-1795). Based on this core heparin binding motif, a 10-amino acid GAG binding peptide (GBP) spanning residues 32-41 on ECP has been proved to possess GAG recognition activity.

Carcinoma also named as epithelial cancer, is a malignant cancer originating in the ectodermal and endodermal epithelial cells. When epithelial cells are transformed from their native characteristics and programmed in the process known as epithelial-to-mesenchymal transition (EMT) to migrate to the secondary locations due to loss of cell adhesion, abnormal expression of cell surface GAGs takes place to alter various growth factor-receptor binding activities. Currently, accumulating evidences indicate that altered expression of proteoglycans and GAGs takes place in many cancer cells (Table 1). For example, syndecan-1 is overexpressed in myeloma patients and correlates with poor prognosis. Syndecan-2 is often overexpressed in colon carcinoma. Syndecan-4 is upregulated in hepatocellular carcinoma and malignant mesothelioma with increasing tumor cell proliferation. Glypican-1 is overexpressed in breast and brain cancers (gliomas) while glypican-3 (Gpc 3) is overexpressed in liver cancers, lung squamous cell carcinoma, metastatic melanoma, Merkel cell carcinoma, and ovarian clear cell adenocarcinoma.

TABLE 1
Alterations of GAGs and Proteoglycan Expression in Cancer Cells

| Cancer | HSGAGs | CSGAGs | Other GAGs |
|---|---|---|---|
| Lung | Sdc-1↑, Sdc-2↑, | CS↑, C4S↑, | ESM-1↑ |
| | Sdc3↑, Sdc-4↑ | C6S↑ | |
| Breast | Sdc-1↑, Gpc-1↑ | VCAN↑, CS↑ | DS↑, HAS-2↓ |
| Colon | Sdc-2↑, PLC↑, | VCAN↑ | DS↓ |
| | 6-O-ST↑ Sdc-1↓, | | |
| | Sdc-4↓ | | |
| Melanoma | Sdc-1↑, Gpc-3↑ | VCAN↑ | |
| Pancreatic | Sdc-2↑ | VCAN↑, C4S, | DS↑ |
| | | C6S↑ | |
| Gastric | Gpc-3↑ | CS↑ | DS↑, HA↑ |
| Prostate | Sdc-2↑, HS, 2-O-ST↑ | VCAN↑, CS↑ | DS↑ |

Sdc (syndecan), Gpc (glypican), PLC (perlecan), HS (Heparan sulfate), CS (chondroitin sulfate), C4S (condroitin-4 sulfate), C6S (chondroitin-6-sulfate), VCAN (versican), DS (dermatan sulfate), ESM-1 (endocan), HA (hyaluronan), HAS (hyaluronan synthase), ST (Sulfotransferase)

### BRIEF DESCRIPTION OF THE DRAWINGS

FIG. 1 shows electrophoresis pattern of indicated concentration of GBP binding to various glycosaminoglycans (GAGs). Fluorophore-assisted carbohydrate electrophoresis (FACE) is carried out by pre-mixing (A) AMAC-labeled LMWH, chondroitin sulfate (CS), and dermatan sulfate (DS) and (B) AMAC-labeled LMWH, de-2-O-sulfated heparin, de-6-O-sulfated heparin, and N-acetyl-de-O-sulfated heparin (0.33 nmol) with indicated fold molar excess of GBP before incubated with or without peptide in PBS at 25° C. for 15 min. The reaction products are separated on a 1% agarose gel. The reacted probe and protein are shown above the gel. Relative intensity (%) of free probe and the unlabeled competitors are shown at the bottom of the gel.

FIG. 2 shows expression of heparan sulfate (HS) in the adenocarcinoma of multiple cancer tissues. Immunohistochemical (IHC) localization of HS is carried out by Supersensitive non-biotin HRP detection system with an anti-HS antibody. Representative IHC staining patterns of HS (brown color) are detected in lung (A), colon (B), stomach (C), pancreas (D), prostate (E) and rectum (F). Nuclei are stained with hematoxylin counterstain in blue. (Magnification: A, B, C, D, E and F, 400×)

FIG. 3 shows targeting of eGFP-GBP in the adenocarcinoma of multiple cancer tissues. IHC localization of eGFP-GBP is carried out by Supersensitive non-biotin HRP detection system with an anti-eGFP antibody. Representative IHC staining patterns of eGFP-GBP (brown color) are detected in lung (A), colon (B), stomach (C), pancreas (D), prostate (E) and rectum (F). Anti-eGFP antibody without eGFP-GBP incubation detected in lung cancer tissue is used as a negative reaction (G). Nuclei are stained with hematoxylin counterstain in blue. (Magnification: A, B, C, D, E, F and G 400×)

FIG. 4 shows expression of HS in squamous cell carcinoma of multiple cancer tissues. IHC localization of HS is carried out by Supersensitive non-biotin HRP detection system with an anti-HS antibody. Representative IHC staining patterns of HS (brown color) are detected in lung (A), esophagus (B) and skin (C). Nuclei are stained with hematoxylin counterstain in blue. (Magnification: A, B and C 400×)

FIG. 5 shows targeting of eGFP-GBP in squamous cell carcinoma of multiple cancer tissues. IHC localization of eGFP-GBP is carried out by Supersensitive non-biotin HRP detection system with an anti-eGFP antibody. Representative IHC staining patterns of eGFP-GBP (brown color) are detected in lung (A), esophagus (B) and skin (C). Nuclei are stained with hematoxylin counterstain in blue. (Magnification: A, B and C 400×)

FIG. 6 shows expression of HS and targeting of eGFP-GBP in hepatocellular carcinoma of liver cancer tissues. IHC localization of HS and eGFP-GBP are carried out by Supersensitive non-biotin HRP detection system. Representative IHC staining patterns of HS (A) and eGFP-GBP (B) (brown color) are detected in liver. Nuclei were stained with hematoxylin counterstain in blue. (Magnification: A and B 400×)

FIG. 7 shows immunoreactivity of HS expression and eGFP-GBP binding in cancer progression. IHC staining patterns (dark brown color) of HS (A to C) and GBP (D to F) by anti-HS antibody and anti-eGFP antibody, respectively, are detected in cancer adjacent normal lung tissue (A and D), cancer adjacent normal lung tissue (hyperplasia of stroma) (B and E), lung adenocarcinoma (C and F). Nuclei are stained with hematoxylin counterstain in blue. (Magnification: B, C, D, E, F and G 400×)

FIG. 8 shows expression of HS in neoplastic lung tissues. IHC localization of HS is carried out by Supersensitive non-biotin HRP detection system with anti-HS antibody. Representative IHC staining patterns of HS (brown color) are detected in normal lung tissue (A), low-graded adenocarcinoma (B), high-graded adenocarcinoma (C), low-graded squamous cell carcinoma (D), high-graded squamous cell carcinoma (E), bronchioloalveolar carcinoma (F) and large cell carcinoma (G). Nuclei are stained with hematoxylin counterstain in blue. (Magnification: A, B, C, D, E 400×)

FIG. 9 shows expression of CS in neoplastic lung tissues. IHC localization of CS is carried out by Supersensitive non-biotin HRP detection system with anti-CS antibody. Representative IHC staining patterns of CS (brown color) are detected in normal lung tissue (A), low-graded adenocarcinoma (B), high-graded adenocarcinoma (C), low-graded squamous cell carcinoma (D), high-graded squamous cell carcinoma (E), bronchioloalveolar carcinoma (F) and large cell carcinoma (G). Nuclei are stained with hematoxylin counterstain in blue. (Magnification: A, B, C, D, E 400×)

FIG. 10 shows tissue targeting of eGFP-GBP in neoplastic lung tissues. IHC localization of eGFP-GBP is carried out by Supersensitive non-biotin HRP detection system with anti-eGFP antibody. Representative IHC staining patterns of eGFP-GBP (brown color) are detected in e normal lung tissue (A), low-graded adenocarcinoma (B), high-graded adenocarcinoma (C), low-graded squamous cell carcinoma (D), high-graded squamous cell carcinoma (E), bronchioloalveolar carcinoma (F) and large cell carcinoma (G). Nuclei are stained with hematoxylin counterstain in blue. (Magnification: A, B, C, D, E 400×)

FIG. 11 shows tissue targeting of eGFP-GBP in mouse cancer model. IHC localization of GBP is carried out using Supersensitive non-biotin HRP detection system with anti-eGFP antibody. Representative IHC staining patterns of eGFP-GBP (brown color) are detected in bronchial-epithelial (A), intestinal villi (C), liver (E), kidney (G) and cancer (I) tissue 1 h post-intravenous (i.v.) injection. A control section of lung (B), intestinal (D), liver (F), kidney (H) and cancer (J) tissue subjected to eGFP injection are processed in parallel. Nuclei are stained blue with hematoxylin counterstain. (Magnification: A, B, C, D, E, F, G, H, I, J 100×)

FIG. 12 shows in vivo distribution of magnetic nanoparticle (MNP) in CT-26 tumor mice. Magnetic resonance image (MRI) axial image of kidney (A-C) and CT-26 tumor (D-F) (yellow arrow) with injection of 150 μl MNP (0.06 emu/g) are taken at 30 min and 21 h after injection intravenously. ddH₂O is set as a positive control for normalization of bright density (white sphere). MRI signals representing MNP targeting to kidney (G) and CT-26 tumor (H) at 30 min and 21 h after injection intravenously are quantified.

FIG. 13 shows in vivo targeting of MNP-GBP to CT-26 tumor. MRI axial images of mouse kidney (A, yellow arrow) and tumor (D, yellow arrow) before intravenously injection with 150 μl MNP-GBP (0.06 emu/g) are monitored. MRI axial images of mouse kidney (B, white arrow) and tumor (E, white arrow) is taken at 0.5 h after injection. The images of mouse kidney (C, white arrow) and tumor (F, white arrow) at 24 h after injection are also taken. ddH₂O is set as a positive control for normalization of bright density (white sphere). MRI contrast of the kidney tissue (G) and tumor (H) at 24 h after injection of MNP-GBP. (I) Photomicrographs of tumor tissue injected with MNP-GBP at 24 h with magnifications of 400× and ferrous iron of MNP-GBP accumulation (black dot) is detected using Prussian blue staining.

### SUMMARY OF THE INVENTION

The present invention relates to a method of delivering an agent to a cancer cell, comprising: (a) obtaining a peptide that selectively binds to a cancer cell, wherein the peptide comprises a cancer targeting motif having the amino acid sequence of SEQ ID NO: 1, wherein the peptide is attached or fused to an agent that one desires to target to a cancer cell; and (b) exposing the peptide to a population of cells suspected of containing cancer cells. The present invention also relates to a kit for applying the above method, comprising a peptide that binds to a cancer cell, wherein the peptide comprises a cancer targeting motif having the amino acid sequence of SEQ ID NO: 1.

### DETAILED DESCRIPTION OF THE INVENTION

In the present invention, screening of various normal and cancer cell lines demonstrats that GBP has preference in binding to high metastasis adenocarcinoma, presumably due to different sulfated GAG expression levels on normal and tumor cells. GBP has also been demonstrated to probe lung tumor types, especially adenocarcinoma and squamous cancer by tissue microarrays. In addition, GBP shows in vivo tumor targeting activity, suggesting its feasibility in targeting to epithelial cancers.

The terms used in the description herein will have their ordinary and common meaning as understood by those skilled in the art, unless specifically defined otherwise. As used throughout the instant application, the following terms shall have the following meanings:

The term “GBP” refers to a 10-amino acid glycosaminoglycan (GAG) binding peptide with the amino acid sequence of NYRWRCKNQN (SEQ ID No: 1).

Thus, the present invention provides a method of delivering an agent to a cancer cell, comprising: (a) obtaining a peptide that binds to a cancer cell, wherein the peptide comprises a cancer targeting motif having the amino acid sequence of SEQ ID NO: 1, wherein the peptide is attached or fused to an agent that one desires to target to a cancer cell; and (b) exposing the peptide to a population of cells suspected of containing cancer cells. In a preferred embodiment, the population of cells is in a mammalian subject. Preferably, the mammalian subject is a human subject. In a preferred embodiment, the population of cells is selected from the group consisting of a thin section of a tissue, a thick section of a tissue, blood and circulating tumor cells. In a preferred embodiment, the cancer is epithelial cancer. Preferably, the epithelial cancer is adenocarcinoma or squamous cell carcinoma. More preferably, the adenocarcinoma is high metastasis adenocarcinoma. More preferably, the adenocarcinoma is selected from lung adenocarcinoma and colon adenocarcinoma. In a preferred embodiment, said method further comprises detecting cancer cells in said population. In a preferred embodiment, said method further comprises diagnosing cancer. In a preferred embodiment, the agent is a therapeutic agent or an imaging agent. Preferably, the agent and peptide are administered to an individual having or suspected of having cancer to treat or image. In a preferred embodiment, the therapeutic agent is a drug, a chemotherapeutic agent, a radioisotope, a pro-apoptosis agent, an anti-angiogenic agent, a survival factor, an anti-apoptotic agent, an enzyme, a hormone, a hormone antagonist, a cytokine, a cytotoxic agent, a cytocidal agent, a cytostatic agent, a growth factor, a peptide, a protein, an antibiotic, an antibody, a Fab fragment of an antibody, a hormone antagonist, a nucleic acid, an antigen, a virus, a bacteriophage, a bacterium, a liposome, a microparticle, a magnetic bead, a microdevice, a yeast cell, a mammalian cell, a cell or an expression vector. Preferably, the agent is an anti-angiogenic agent selected from the group consisting of thrombospondin, angiostatin5, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-B, thrombospondin, 2-methoxyoestradiol, proliferin-related protein, carboxiamidotriazole, CM 101, Marimastat, pentosan polysuiphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, Docetaxel, polyamines, a proteasome inhibitor, a kinase inhibitor, a signaling peptide, accutin, cidofovir, vincristine, bleomycin, AGM-1470, platelet factor 4 and minocycline. In another embodiment, the agent is a cytokine selected from the group consisting of interleukin 1 (IL-1), IL-2, IL-5, IL-10, IL-11, IL-12, IL-18, interferon-γ (IF-γ), IF-α, IF-β, tumor necrosis factor-α (TNF-γ), or GM-CSF (granulocyte macrophage colony stimulating factor). In a preferred embodiment, the imaging agent is a tracing cargo selected from the group consisting of fluorescence tag, chemiluminescence protein, radioisotope, and magnetic nanoparticle. Preferably, the imaging agent is a radioisotope selected from the group consisting of astatine-211, carbon-14, chromium-51, chlorine-36, cobalt-57, cobalt-58, copper-67. En 152, gallium-67, hydrogen-3, iodine-123, iodine-125, iodine-131, indium-111, iron-59, phosphorus-32, rhenium-186, rhenium-188, selenium-75, sulphur-35, technicium-99m and yttrium-90. In a more preferred embodiment, the peptide that binds to a cancer cell is SEQ ID NO: 1.

The present invention also provides a kit for applying the method described above, comprising a peptide that binds to a cancer cell, wherein the peptide comprises a cancer targeting motif having the amino acid sequence of SEQ ID NO: 1. Preferably, the peptide is SEQ ID NO: 1.

### EXAMPLES

The examples below are non-limiting and are merely representative of various aspects and features of the present invention.

### Example 1

### Materials and Methods

### Fluorescence-Assisted Carbohydrate Electrophoresis (FACE)

Carbohydrate were labeled with 2-aminoacridone (AMAC) (Invitrogen, catalog no. A6289) according to previous study (Calabro, A., Benavides, M., Tammi, M., Hascall, V. C., and Midura, R. J. (2000) Microanalysis of enzyme digests of hyaluronan and chondroitin/dermatan sulfate by fluorophore-assisted carbohydrate electrophoresis (FACE), Glycobiology 10, 273-281). Briefly, fifty micrograms GAGs were dried by freeze-drying machine, and then incubated with 40 μl 1.25 M 2-aminoacridone (AMAC)/85% DMSO/15% acetic acid at 25° C. for 15 min. Forty microliters of 1.25 M sodium cyanoborohydride (NaBH₃CN) was added and incubated at 37° C. for 16 h. Afterwards, 720 μl 99% ice-cold ethanol was added at −20° C. for 15 min, then centrifugation at 11,000×g at 4° C. for 5 min. The supernatant was carefully discarded and freeze dried by Spin Vacuum and Freezer (Coolsafe™). The dried pellet was resolubilized with appropriate volume (frequently 20 μL or 50 μL) of sterile deionized water according to the intensity of labeled probe. The prepared AMAC labeled probes were stored at −80° C. and prevent from light.

The AMAC labeled probes and proteins were mixed and incubated at 25° C. for 15 min. The complex then loaded onto 1% agarose gels and electrophoresed in the buffer containing 40 mM Tris-acetic acid, 1 mM EDTA, pH 8.0 for 20 to 30 minutes. This examination was performed in the dark or red light to prevent from light. The AMAC labeled probe was observed under UV light and scanned by transilluminator (ONLY Science Co., ltd).

### Cell Culture

Table 2 is a list of cell lines. Cells were cultured in medium (Gibco, Invitrogen, USA) supplemented with heat-inactivated 10% (v/v) fetal bovine serum (FBS) (Gibco, Invitrogen, USA), and 1% (v/v) Glutamine-Penicillin-Streptomycin (biosera). Cells were grown on 100-mm dishes and incubated at 37° C. in 5% CO₂.

TABLE 2
List of all tested cell lines

| | | | | |
|---|---|---|---|---|
| Cell line | Type | Morphology | Medium | Metastasis |

Human normal lung cell lines

| | | | | |
|---|---|---|---|---|
| Beas-2B | Bronchus | Epithelial | RPMI1640 | Non |
| MRC-5 | Fetal | fibroblast | DMEM | Non |
| | fibroblast | | | |

Human lung cancer cell lines

| | | | | |
|---|---|---|---|---|
| A549 | Adenocarci- | Epithelial | DMEM | High |
| | noma | | | |
| CL1-0 | Adenocarci- | Epithelial | RPMI1640 | Low |
| | noma | | | |
| CL1-3 | Adenocarci- | Epithelial | RPMI1640 | High |
| | noma | | | |
| CL1-5 | Adenocarci- | Epithelial | RPMI1640 | High |
| | noma | | | |
| CL3 | Adenocarci- | Epithelial | RPMI1640 | High |
| | noma | | | |
| PC9 | Adenocarci- | Epithelial | RPMI1640 | High |
| | noma | | | |
| H157 | Squamous cell | Epithelial | RPMI1640 | High |
| | carcinoma | | | |
| H460 | Large cell | Epithelial | RPMI1640 | Extremely |
| | carcinoma | | | high |
| H1299 | Non-small cell | Epithelial | RPMI1640 | High |
| | lung carcinoma | | | |
| | cell | | | |

Human digestive tract cancer cell lines

| | | | | |
|---|---|---|---|---|
| AGS | Gastric | Epithelial | F-12K | Low |
| | adenocarci- | | | |
| | noma | | | |
| Caco-2 | Colon | Epithelial | MEM | Con |
| | adenocarci- | | | |
| | noma | | | |
| HCT-116 | Colon | Epithelial | McCoy's | High |
| | adenocarci- | | 5A | |
| | noma | | | |
| HT-29 | Colon | Epithelial | DMEM | Low |
| | adenocarci- | | | |
| | noma | | | |
| HepG2 | Hepatocellular | Epithelial | DMEM | Low |
| | carcinoma | | | |

Mouse digestive tract cancer cell lines

| | | | | |
|---|---|---|---|---|
| CT-26 | BALB/c colon | Epithelial | RPMI-1640 | High |
| | adenocarci- | | | |
| | noma | | | |

### Flow Cytometry

Cells (2.0×10⁵ cells/well) were plated into 6-well plates and cultured in medium. After 24 h, cells were incubated with fluorescence-labeled peptides dissolved in medium for 1 h. Cells were harvested, washed and suspended in PBS. Treated cells were subjected to fluorescence analysis on a FACScalibur flow cytometer (FACS, BD Biosciences, Franklin Lakes, N.J.) with 488 nm excitation and collected the emission between 515-545 nm.

### Immunohistochemistry Staining

The 5 μm slides were dried in the oven at 60° C. for 1 h. Briefly, the sections were deparaffinized with xylene (J. T. Baker Phillipsburg, N) for 15 min and rehydrated in graded ethanol solutions. Endogenous peroxidase activity was blocked by incubating the sections in a 3% hydrogen peroxide solution in distilled water for 10 min. The sections were then washed and placed in phosphate buffer for 10 min. Antigen retrieval was achieved by heat treatment using 10 mM citrate buffer solution pH 6.0 or by 0.1% trypsin treatment for 5 min (for 10E4). After the slides were blocked in 3% BSA solution for 1 h and incubated with 2 μM eGFP-GBP fusion protein for 2 h (for eGFP), these sections were then incubated with 10E4 antibody (1:200 dilution) and eGFP antibody (1:200 dilution) at 4° C. overnight, individually. These sections were incubated with super enhancer buffer for 20 min and incubated in second antibody polymer HRP conjugate broad sepectrum for 30 min at room temperature following the wash with phosphate buffer three times. The color was developed by incubation with 3,3′-diaminobenzidine (DAB, 0.2 mg/ml, Pierce, Rockford, Ill., USA) for 3 min and then counterstained with Mayer's hematoxylin for 1 min. Each slide was then soaked with 95% alcohol and 100% alcohol for dehydration followed by soaking with xylene for 15 minutes and covering the slides.

### Scoring of Immunohistochemistry Staining

Every tumor was given a score according to the intensity of the nuclear or cytoplasmic staining (no staining=0; weak staining=1; moderate staining=2; strong staining=3) and the extent of stained cells (0-10%=0; 11-50%=1; 51-80%=2; 81-100%=3). The final immunoreactive score was determined by multiplying the intensity and extent of positivity scores of stained cells, with the minimum score of 0 and a maximum score of 9.

### Animal Model

Adult female Balb/c mice were purchased from, and maintained at, the National Laboratory Animal Center, Taiwan. Mouse colon carcinoma CT-26 cell line was purchased from ATCC (Rockville, Md.). Cells are cultured in medium containing 10% heat-inactivated fetal bovine serum at 37° C. in a humidified atmosphere containing 5% CO₂. Briefly, four to six week-old BALB/c mice are injected subcutaneously with 5×10⁵ CT-26 cells (in 100 μl). As tumor developed to about (8-9)±1 mm³ volume, the mice were separated into two groups and injected with 5 nmol of enhanced green fluorescence protein (eGFP) or eGFP-GBP through their tail veins. All animals were asphyxiated with CO₂, 1 h after injection. Lung, trachea, kidney, intestine and tumor tissues were removed and immediately fixed in 10% neutral-buffered formaldehyde. The tissue samples were processed by standard methods to prepare paraffin wax-embedded block samples (Fan, T. C., Fang, S. L., Hwang, C. S., Hsu, C. Y., Lu, X. A., Hung, S. C., Lin, S. C., and Chang, M. D. (2008) Characterization of molecular interactions between eosinophil cationic protein and heparin, The Journal of biological chemistry 283, 25468-25474). The blocks were sectioned into 6 μm slices and were examined using a Super Sensitive Non-Biotin HRP Detection System (BioGenex Laboratories, San Ramon, Calif.) as previously described (Fan, T. C., Fang, S. L., Hwang, C. S., Hsu, C. Y., Lu, X. A., Hung, S. C., Lin, S. C., and Chang, M. D. (2008) Characterization of molecular interactions between eosinophil cationic protein and heparin, The Journal of biological chemistry 283, 25468-25474). The slices were also visualised by light microscope (Zeiss-Axioplan, Germany).

### MRI Assay

As tumor developed to about (8-9)±1 mm³ volume, each mouse is injected intravenously (i.v.) with 150 μl MNP-GBP (0.06 emu/g) followed by MRI scout scans to accurately position the mouse inside tumor. The magnetic nanoparticles are dextran-coated Fe₃O₄ nanoparticles (GABC Co.), and mean diameter is 52 nm. The saturated magnetization of the magnetic fluid containing magnetic nanoparticles is 0.06 eum/g. The peptide-magnetic nanoparticles homogeneously dispersed in PBS were injected into a mouse from tail vein. The volume of magnetic fluid injected into each mouse is 150 μl. After injection, locations of MNPs were detected with magnetic resonance images (MRIs). MRI examinations were performed on a 7-Tesla system before and one day after the mouse received the injection. For MR scanning, the mice were anaesthetized with 0.5 ml of ketamine and 0.5 ml of rompun. Multiple transverse images are obtained using fat-saturated 3D gradient echo pulse sequence (Turbo FLASH) encompassing the entire aorta, TR=5.17 ms; TE=2.49 ms; flip angle=10°; FOV=256 mm; slice thickness=2 mm; matrix=256×256; voxel size=1×1×2 mm; number of averages=10. In-vivo MR images of the cross section through the abdominal aorta of a control rat and an injected mouse.

### Statistical Analysis

All data were shown as mean±standard deviations (SD) with n, was the number of experiments performed. To compare two means, statistical analysis was performed with unpaired Student's t test using GraphPad Prism v 4.02 (GraphPad Software, USA). One-way analysis of variance (ANOVA) was used to test for differences among multiple treatments, followed by the Dunnett's test. P value less than 0.05 was considered statistically significant.

### Results

### Tumor Cell Binding Activity of GBP

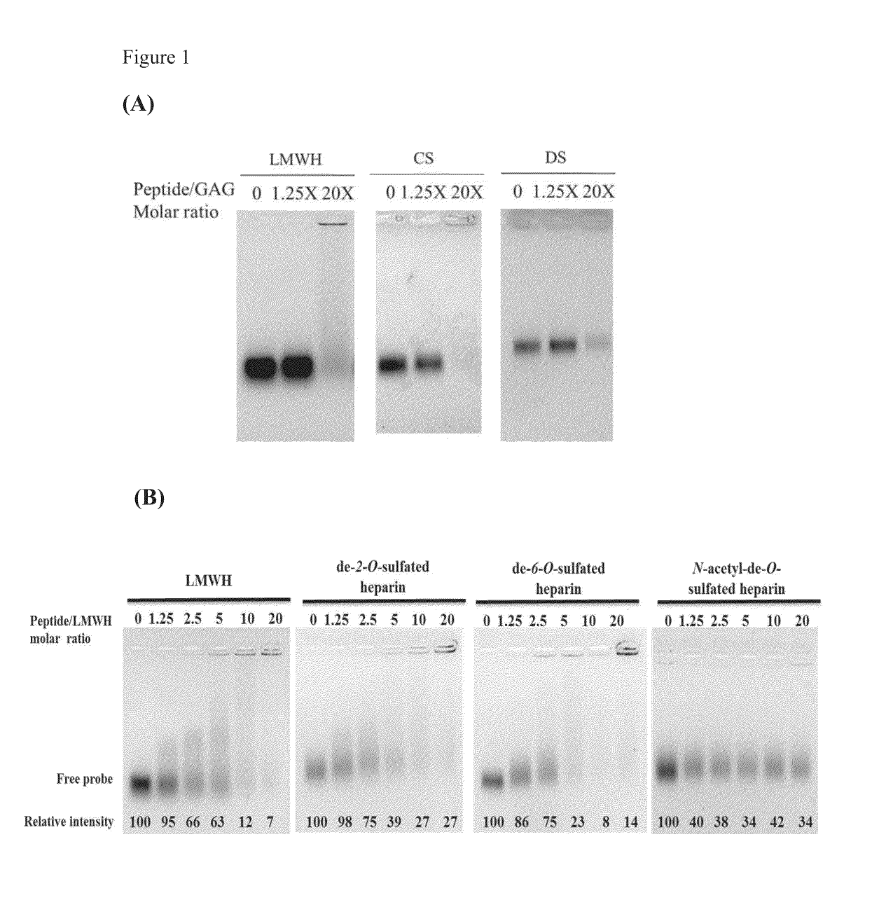

GBP probed universal glycosaminoglycans including low molecular weight heparin, chondroitin sulfate and dermatan sulfate (FIG. 1A). Moreover, GBP identified sulfated HS, especially preferring O-sulfated heparin (FIG. 1B). The most common structure found in heparin was a trisulfated disaccharide at 2-O position of IdoA and 2-N, 6-O positions of GlcNAc. Monoclonal anti-heparan sulfate (10E4) antibody detected epitope of N-sulfated glucosamine on HS. Thus, 10E4 and FITC-GBP were detected their signals in various normal and cancer cell lines to quantitatively determine a correlation between the binding of GBP and expression of HSPGs (Table 2) by flow cytomtery. As shown in Table 3, GBP showed higher binding level to cancer cell lines such as human lung adenocarcinoma H460, lung adenocarcinoma CL1-3, lung large cell carcinoma PC9, human AGS gastric adenocarcinoma cells, human HCT-116 colorectal carcinoma cells, human HepG2 hepatocellular carcinoma cells, and mouse CT-26 colon adenocarcinoma cells. Moreover, GBP also bound to CL3, A549, H157, CL1-5, Caco-2 and HT-29. Compared with the binding level of GBP in Beas-2B cell, GBP bound much more to cancer cell lines (Table 3). In addition, GBP showed higher binding level in higher metastasis tumors than lower ones in lung adenocarcinoma and colon adenocarcinoma, suggesting that GBP preferred attaching to abnormally increased expression of GAGs on selective cancer cell surface during tumor progression. Although the expression level of N-sulfated glucosamine on HS in normal lung Beas-2B cells and MRC-5 cells was higher than lung cancer cell lines, all lung cancer cell lines were recognized by GBP more significantly than normal lung cell lines. Taken together, these observations suggested that GBP showed a significant selectivity in binding to high metastatic epithelial cancers. Correlation between HS profiles (N-sulfated glucosamine) and GBP binding signals suggested that expression patterns between normal and tumor cells were somehow discrepant, possibly due to different regulatory effects of GAG catalytic enzymes such as sulfatase in tumorigenesis progression.

TABLE 3
Cell surface HSPG profiles and GBP binding activity

| | | |
|---|---|---|
| Cell lines | FITC-GBP binding | HSPGs* |

Human normal lung tissue cell lines

| | | |
|---|---|---|
| Beas-2B | 8.10 ± 0.79 | 5.89 ± 1.39 |

Human lung cancer cell lines

| | | |
|---|---|---|
| H460 | 28.6 ± 0.81 | 1.81 ± 0.06 |
| CL1-3 | 27.31 ± 4.40 | 8.13 ± 1.92 |
| PC9 | 22.2 ± 4.48 | 2.52 ± 0.58 |
| CL3 | 19.51 ± 0.96 | 5.12 ± 0.3 |
| A549 | 16.15 ± 1.53 | 2.84 ± 0.06 |
| H157 | 16.14 ± 1.77 | 4.0 ± 2.3 |
| CL1-5 | 14.98 ± 1.11 | 3.91 ± 0.84 |
| CL1-0 | 10.27 ± 1.83 | 2.44 ± 0.27 |
| H1299 | 8.21 ± 0.37 | 2.81 ± 0.37 |

Human digestive tract cancer cell lines

| | | |
|---|---|---|
| AGS | 76.68 ± 3.87 | 3.60 ± 1.27 |
| HT-29 | 11.21 ± 0.61 | 2.39 ± 0.96 |
| Caco-2 | 19.33 ± 0.85 | 4.49 ± 0.58 |
| HCT-116 | 32.97 ± 1.46 | 1.63 ± 0.01 |
| HepG2 | 21.87 ± 4.67 | 3.72 ± 1.27 |

Mouse digestive tract cancer cell lines

| | | |
|---|---|---|
| CT-26 | 21.97 ± 1.5 | 25.37 ± 7.08 |

*10E4 recognizes N-sulfated glucosamine on HS

### Immunohistochemical Screening in Multiple Carcinoma Tissue Arrays

Specimens from 37 cases of multiple organ carcinoma microarrays containing 9 examples of cancers from 16 different tissues (esophagus, stomach, colon, rectum, liver, lung, kidney, breast, uterine cervix, ovary, bladder, lymph node, skin, brain, prostate, pancreas) (BCN9636, US Biomax, Rockville, USA) were used for immunohistochemical (IHC) screening. Monoclonal anti-HS antibody and eGFP-GBP were applied to detect signal in multiple carcinoma tissue arrays. The clinic pathological features of these cases are summarized in Table 4.

TABLE 4
Histological diagnosis of human multiple cancer tissues

| HISTOLOGY | NUMBER |
|---|---|
| Adenocarcinoma | 10 |
| Lung | 2 |
| Colon | 3 |
| Stomach | 3 |
| Pancreas | 2 |
| Squamous cell carcinoma | 6 |
| Lung | 1 |
| Esophagus | 2 |
| Skin | 3 |
| Other type cancer | 21 |
| Astrocytoma | 3 |
| B-cell lymophma | 3 |
| Clear cell carcinoma | 3 |
| Hepatocellular carcinoma | 3 |
| Invasive ductal carcinoma | 3 |
| Transitional cell carcinoma | 3 |
| Serous adenocarcinoma | 3 |
| | 37 |

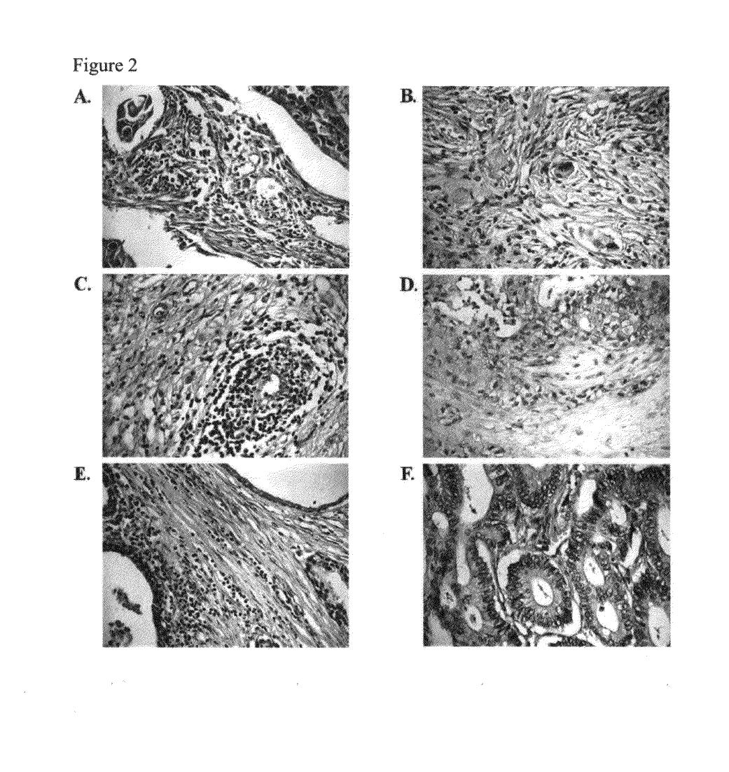

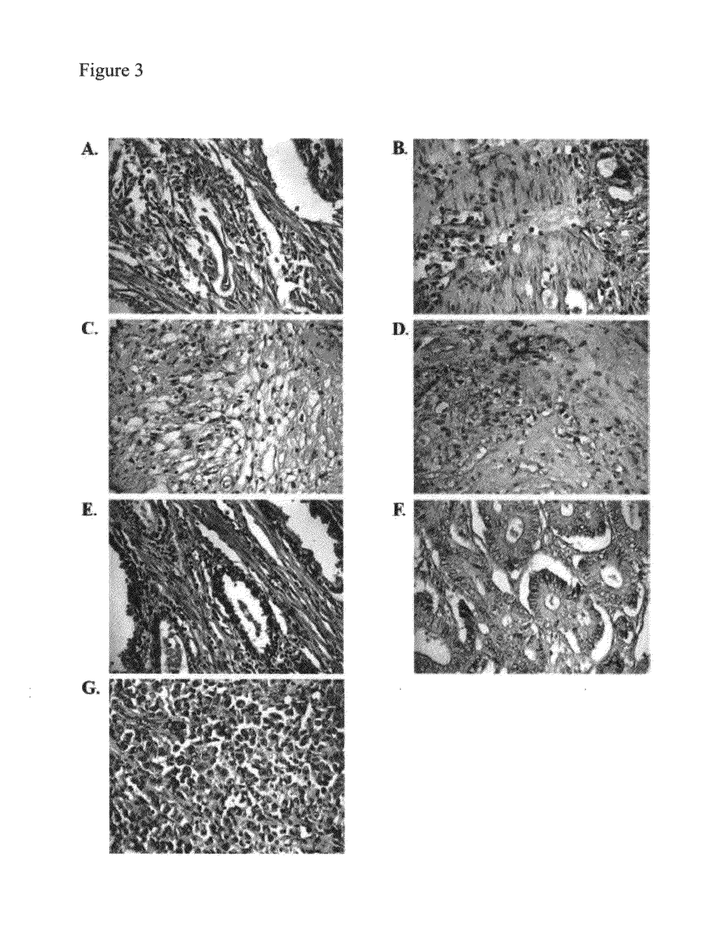

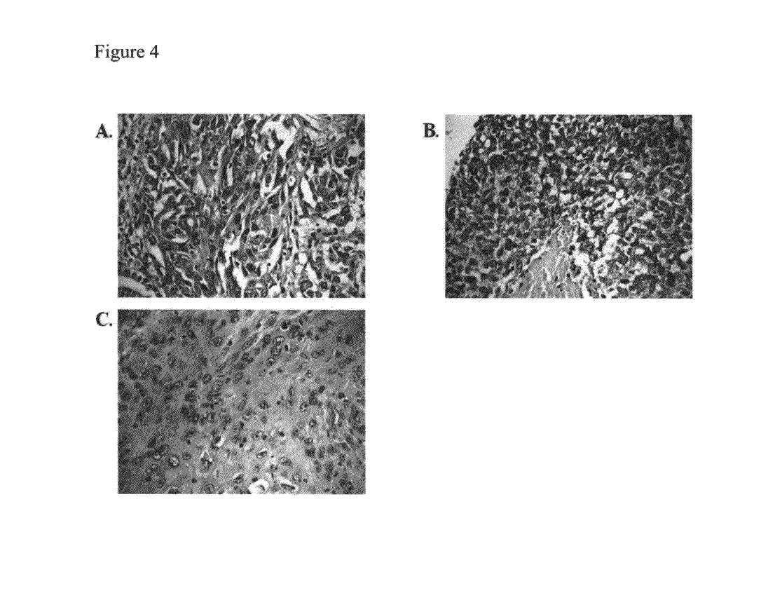

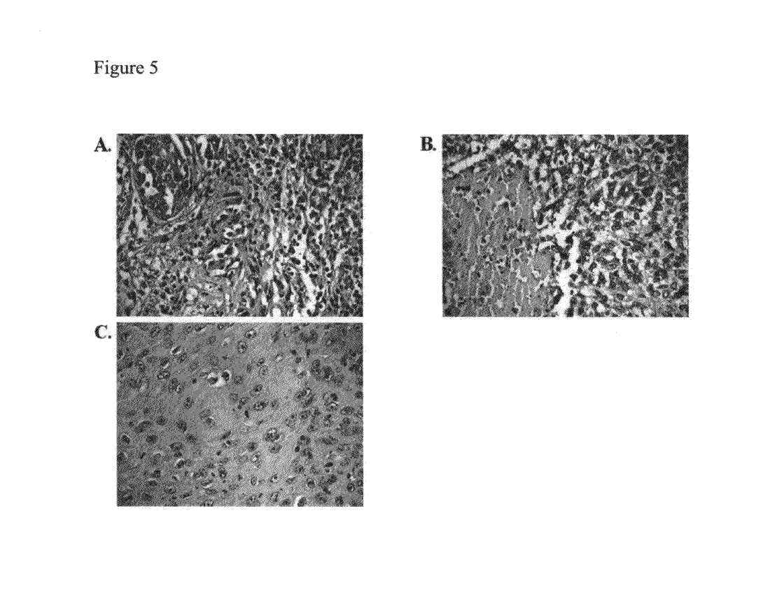

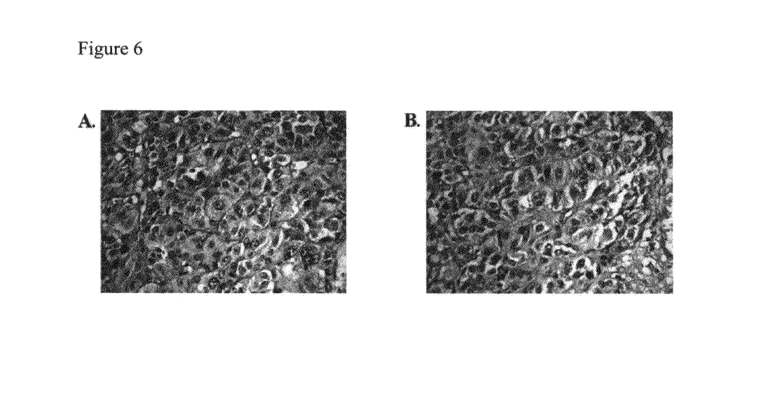

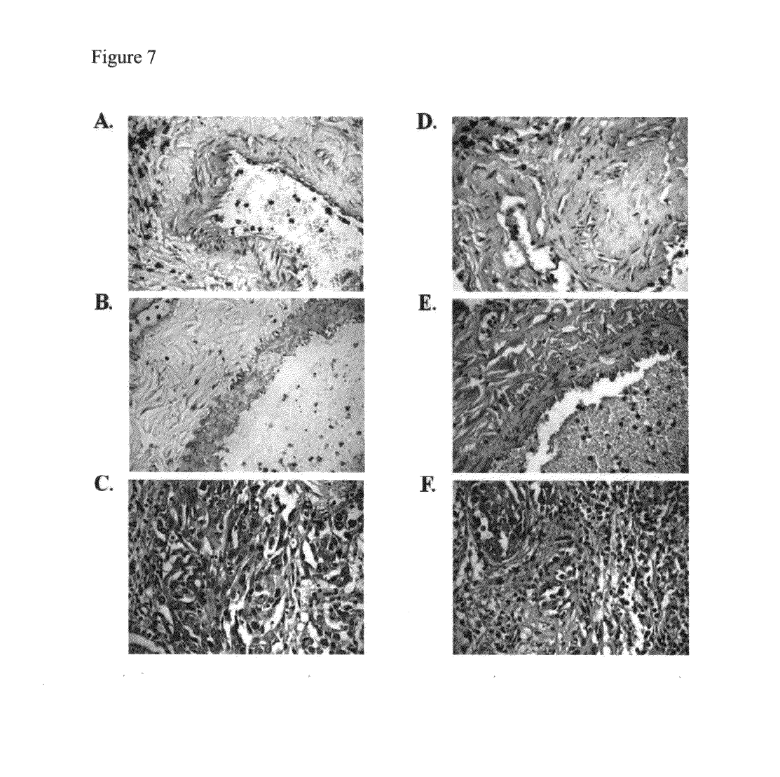

In adenocarcinoma type, HS molecules were highly expressed in lung tissue (FIG. 2A) and less expressed in the colon (FIG. 2B), stomach (FIG. 2C), pancreas (FIG. 2D), prostate (FIG. 2E) and rectum (FIG. 2F). Likewise, eGFP-GBP signal was also obviously observed in lung tissue (FIG. 3A) and obscurely showed in colon (FIG. 3B), stomach (FIG. 3C), pancreas (FIG. 3D), prostate (FIG. 3E) and rectum (FIG. 3F). As for the negative control, anti-eGFP antibody showed no signal (FIG. 3G). In addition, HS molecules were highly expressed in lung squamous cell carcinoma tissue (FIG. 4A) similar to what adenocarcinoma from lung tissue did (FIG. 2A), but squamous cell carcinoma from esophagus (FIG. 4B) and skin (FIG. 4C) tissues only expressed low level of HS. Similarly, eGFP-GBP signal was evidently detected in lung cells (FIG. 5A) but weakly immunolocalized to esophagus (FIG. 5B) and skin (FIG. 5C) tissues. In other cancers, only hepatocellular carcinoma showed weak HS expression (FIG. 6A) and eGFP-GBP signal (FIG. 6B), but astrocytoma, B-cell lymophma, clear cell carcinoma, invasive ductal carcinoma, transitional cell carcinoma and serous adenocarcinoma did not display either signals. Pathologic hyperplasia with an increase in cell numbers in a tissue or organ may be a sign of abnormal or precancerous changes. Surprisingly, both HS expression level (FIG. 7A˜7C) and eGFP-GBP binding intensity (FIG. 7D˜7F) evidently increased along with cancer progression, as illustrated in IHC images of normal tissue (FIGS. 7A and 7D), normal tissue with hyperplasia (FIGS. 7B and 7E), and adenocarcinoma (FIGS. 7C and 7F). In summary, the staining intensities of HS expression level and GBP targeting were obviously stronger in adenocarcinoma and squamous cell carcinoma type from lung tissue than both signals in others, providing a significant clue that GBP could be applied as a novel detection tool to identify early to middle stages of lung cancers.

### Expression Patterns of Glycosaminoglycans in Lung Cancer

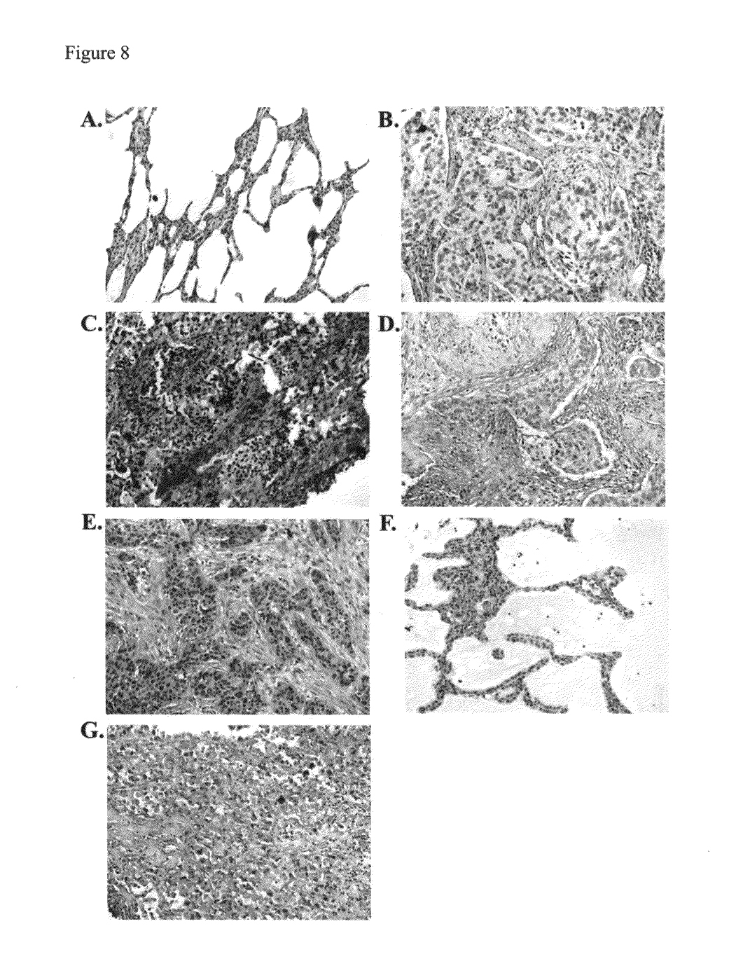

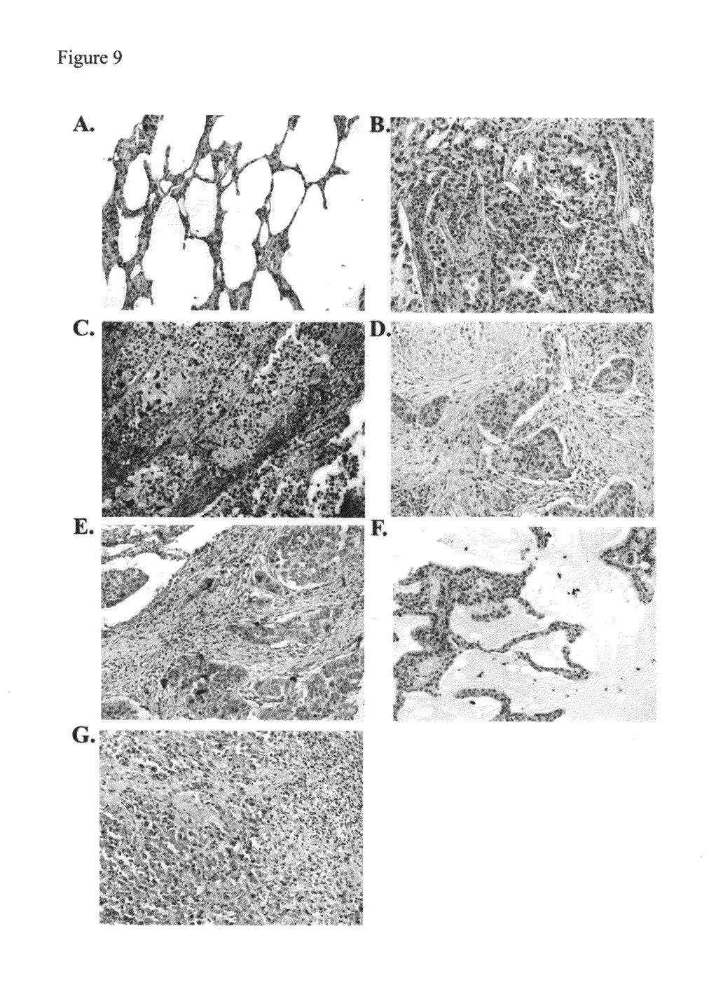

Specimen scores from 61 cases of lung cancers from tissue arrays were used for quantitative evaluation of IHC data. In these 61 cases, the samples included normal lung tissue, adenocarcinoma, squamous cell carcinoma, bronchioloaveloar carcinoma, and large cell carcinoma (Table 5). In tumoral or nontumoral regions, membrane-associated glycosaminoglycans such as HS and CS were expressed at different degree. The expression levels of HS and CS on surface epithelia of normal lung tissue were negative as well as the fibroblasts in the lamina propria (FIGS. 8A and 9A).

TABLE 5
Histological diagnosis of 61 examined human lung tissues

| HISTOLOGY | NUMBER |
|---|---|
| Normal lung tissue | 4 |
| Adenocarcinoma | 22 |
| Bronchioloalveolar carcinoma | 10 |
| Large cell carcinoma | 10 |
| Squamous cell carcinoma | 15 |
| | 61 |

Adenocarcinoma (FIGS. 8B & 8C and 9B & 9C) and squamous cell carcinoma cases (FIGS. 8D & 8E and 9D & 9E) displayed strong stain in HS and CS expression in extracellular membrane. In addition, GBP demonstrated higher recognition toward more aggressive tumor progression (FIGS. 8C & 8E and 9C & 9E) than early stage of cancer (FIGS. 8C & 8E and 9C & 9E). Bronchioloaveolar carcinoma showed mild stain in HS and CS expression on membrane (FIGS. 8F and 9F). The large cell carcinoma showed high HS expression (FIG. 8G) but weak CS expression (FIG. 9G) on membrane. Staining scores of lung cancers demonstrated the IHC results of expression patterns of HS and CS in different types of lung cancer (Table 6): (1) normal lung tissues were stained in a 100% negativity (4/4); (2) adenocarcinoma was in a 27.3% negativity (6/22), in a 59.1% weak or moderate positivity (13/22) and in a 13.6% strong positivity (3/22); (3) squamous cell carcinoma was stained in a 40% weak or moderate positivity (6/15) and in a 60% strong positivity (9/15); (4) bronchioloaveolar carcinoma was stained in a 20% negativity (2/10), in a 80% weak or moderate positivity (8/10); (5) large cell carcinoma was stained in a 90% weak or moderate positivity (9/10) and in a 10% strong positivity (1/10). These results suggested that the comparatively high expression level of HS and CS in adenocarcinoma and squamous cell carcinoma.

TABLE 6
Immunoreactivity scores in lung tissues
represented by HS expression and GBP

| | Anti-HS (10E4) | | | Anti-eGFP (eGFP-GBP) | | |
|---|---|---|---|---|---|---|
| SITE | 0 < IRS < 1 | 1 < IRS < 4 | 4 < IRS < 9 | 0 < IRS < 1 | 1 < IRS < 4 | 4 < IRS < 9 |
| Normal lung tissue | 4 | 0 | 0 | 2 | 2 | 0 |
| (n = 4) | (100%) | (0) | (0) | (50%) | (50%) | (0) |
| Adenocarcinoma | 6 | 13 | 3 | 3 | 12 | 7 |
| (n = 22) | (27.3%) | (59.1%) | (13.6%) | (13.6%) | (54.5%) | (31.9%) |
| Squamous cell carcinoma | 0 | 6 | 9 | 3 | 7 | 5 |
| (n = 15) | (0) | (40%) | (60%) | (20%) | (46.7%) | (33.3%) |
| Bronchioloalveolar carcinoma | 2 | 8 | 0 | 4 | 4 | 2 |
| (n = 10) | (20%) | (80%) | (0) | (40%) | (40%) | (20%) |
| Large cell carcinoma | 0 | 9 | 1 | 0 | 9 | 1 |
| (n = 10) | (0) | (90%) | (10%) | (0) | (90%) | (10%) |

IRS: immunoreactivity score

### Targeting of GBP in Lung Cancer

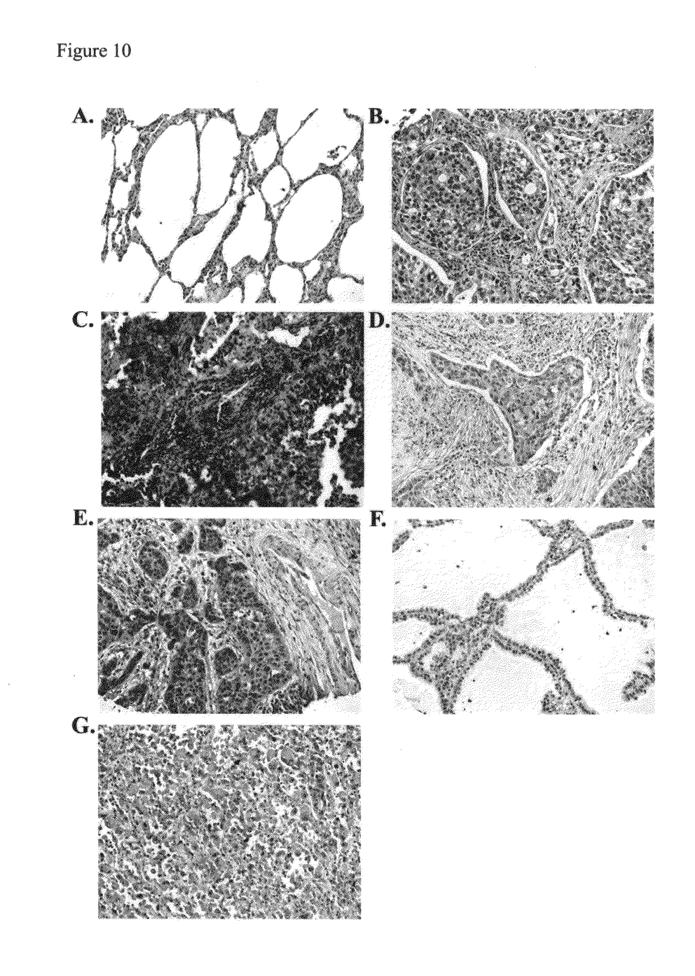

GBP had previously showed high binding to lung cancers in vitro. eGFP-GBP was also assessed for its molecular probing by the lung cancer tissue arrays. eGFP-GBP signal was weakly detected in the endothelia and fibroblasts in the normal lung tissue (FIG. 10A). Similarly, targeting signal of eGFP-GBP displayed extremely strong staining intensities in the adenocarcinoma (FIG. 10B) and squamous cell carcinoma (FIG. 10C). In addition, targeting intensities of eGFP-GBP also showed strong in the bronchioloaveolar carcinoma (FIG. 10D) and large cell carcinoma (FIG. 10E). IHC results of targeting intensity of eGFP-GBP in different types of lung cancers were summarized in Table 6: (1) normal lung tissues were stained in a 50% negativity (2/4) and in a 50% weak positivity (2/4); (2) adenocarcinoma was stained in a 13.6% negativity (3/22), in a 54.5% weak or moderate positivity (12/22) and in a 31.9% strong positivity (7/22); (3) squamous cell carcinoma was stained in a 20% negativity (3/15), 46.7% weak or moderate positivity (7/15) and 33.3% strong positivity (5/15); (4) bronchioloaveolar carcinoma was stained in 40% negativity (4/10), 40% weak or moderate positivity (4/10) and 20% strong positivity (2/10); (5) large cell carcinoma was stained in a 90% weak or moderate positivity (9/10) and 10% strong positivity (1/10). Analysis of these tissue microarrays indicated that targeting intensity of eGFP-GBP was proved again to be similar to the expression patterns of HS and CS in all cases of lung cancers, suggesting that eGFP-GBP preferentially possesses adenocarcinoma and squamous cell carcinoma targeting.

### Probing Activity of GBP in Tumor Animal Model

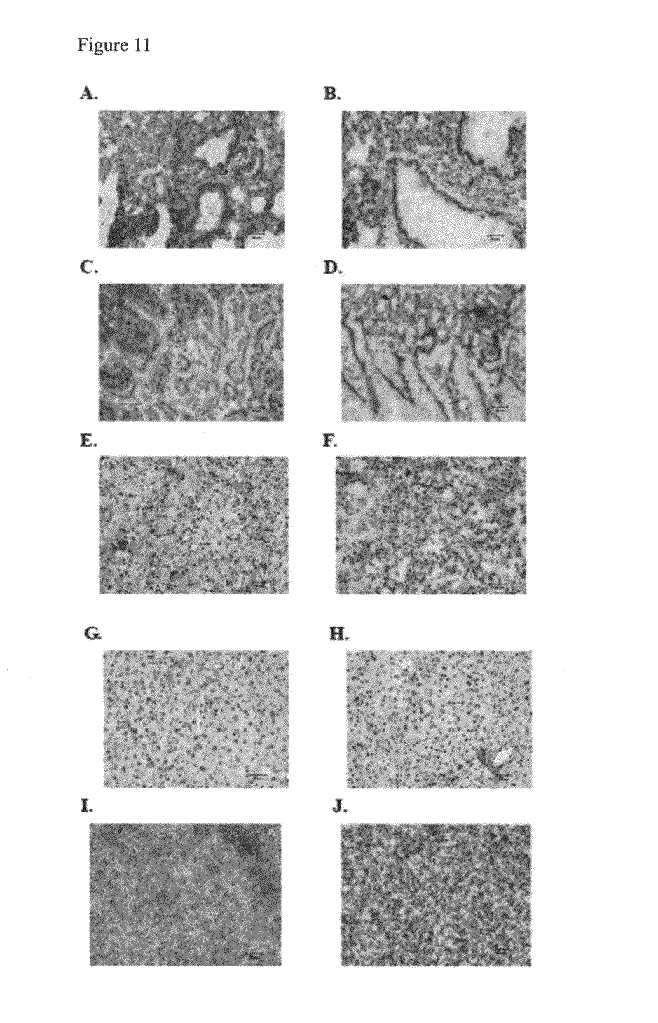

CT-26, a mouse colon adenocarcinoma cell line, showed both high HSPG expression and high GBP binding activity (Table 3). Hence, CT-26 tumor-bearing mouse were chosen for in vivo GBP targeting model. Recombinant eGFP-GBP and eGFP proteins were injected into CT-26 bearing mouse for 1 hour followed by sacrifice and tissue dissection. Tissue sections were verified in vivo targeting activity of eGFP-GBP by IHC staining with anti-eGFP antibody. eGFP-GBP signals were slightly detected in broncho-epithelial (FIG. 11A), intestinal villi (FIG. 11C), kidney (FIG. 11E) liver (FIG. 11G), however, strongly in cancer tissue (FIG. 11I). Kidney and liver were both considered as the main excretory in mammals, which expelled most wastes and toxins to the outside of body. Therefore, most of enthetic protein would be detected in both kidney (FIG. 11E) and liver (FIG. 11G). Amazingly, significantly high amount of eGFP-GBP signal was detected in CT-26 cancer tissue (FIG. 11I). For the negative control (eGFP injection), eGFP signals detected by eGFP antibody were not observed in broncho-epithelial (FIG. 11B), intestinal (FIG. 11D) or CT-26 tumor tissues (FIG. 11J), while clear eGFP signals were also detected in emunctory organs kidney (FIG. 11F) and liver (FIG. 11H) due to rapid transportation through circulation system. These data pointed out that GBP possessed high selectivity to recognize adenocarcinoma cells in mouse model as well.

### Tumor Targeting of MNP-GBP

Magnetic nanoparticle (MNP) is applied for magnetic resonance (MR) imaging analysis, which provided a non-invasive and real-time in vivo monitor on animal model. T2-weighted contrast MR image, also called relaxation enhancement proton-density-weighted MR imaging, revealed proton signals especially on water. Several organs such as kidney, liver and tumor tissues were water-rich and easy to be imaged by T2-weighted MRI system. While dextran-coated magnetic MNP (Fe₃O₄) with 52 nm in diameter bound to aforementioned tissues, a decrease in signal intensity was observed.

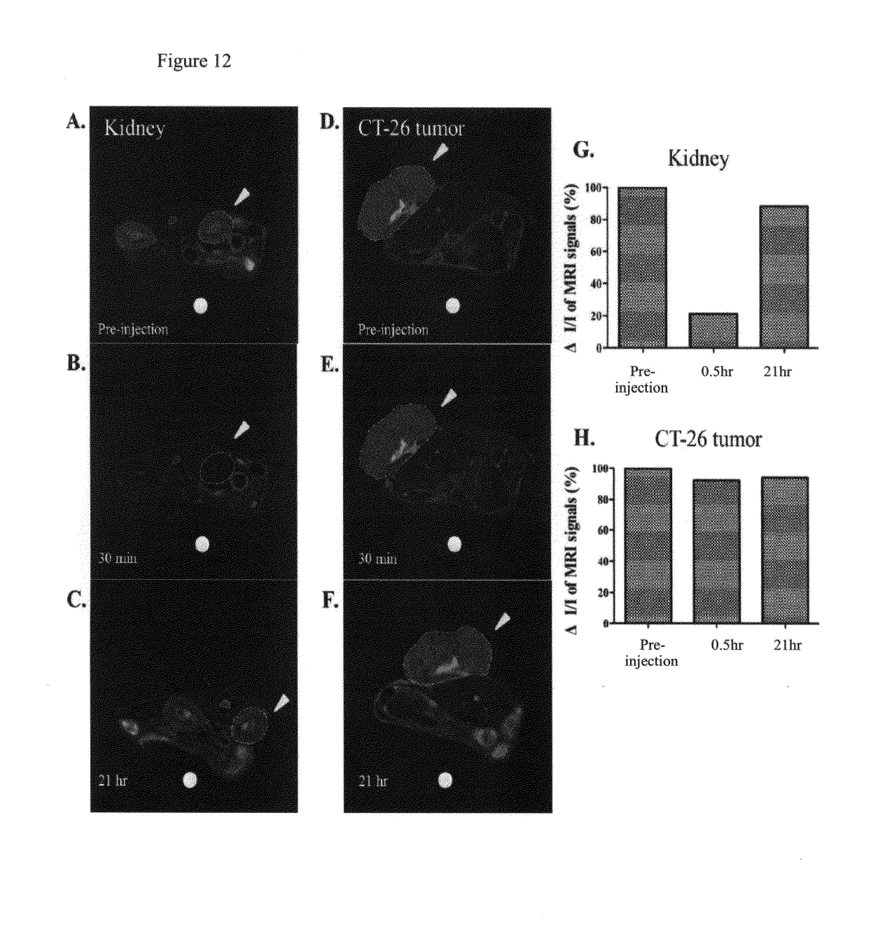

CT-26 was recognized by FITC-GBP (Table 3) and eGFP-GBP fusion protein (FIG. 11I), indicating that MNP-GBP might also be feasible to recognize cancer cells in vivo as well. To test the targeting of MNP in CT26 bearing mouse model, a 12-week-old female Balb/c mouse implanted with CT-26 tumor on the back was injected intravenously with 150 μl MNP (0.06 emu/g). The tumor mouse was respectively imaged at kidney and tumor sites at 0, 0.5 and 21 h after injection of MNP while ddH₂O was set as a positive control for normalization of bright density (FIGS. 12A, 12B and 12C, round signal indicated by yellow arrow). MRI contrast intensity of excretory kidney was enhanced at 0.5 h after injection of MNP (FIG. 12B) but recovered at 21 hour after injection (FIG. 12C). However, MRI contrast intensity of tumor was not enhanced at 0.5 and 21 hour after injection of MNP (FIGS. 12E and 12F), showing that MNP alone did not target the tumor tissue. MRI contrast intensity of kidney and tumor was also quantified in FIGS. 12G and 12H, respectively.

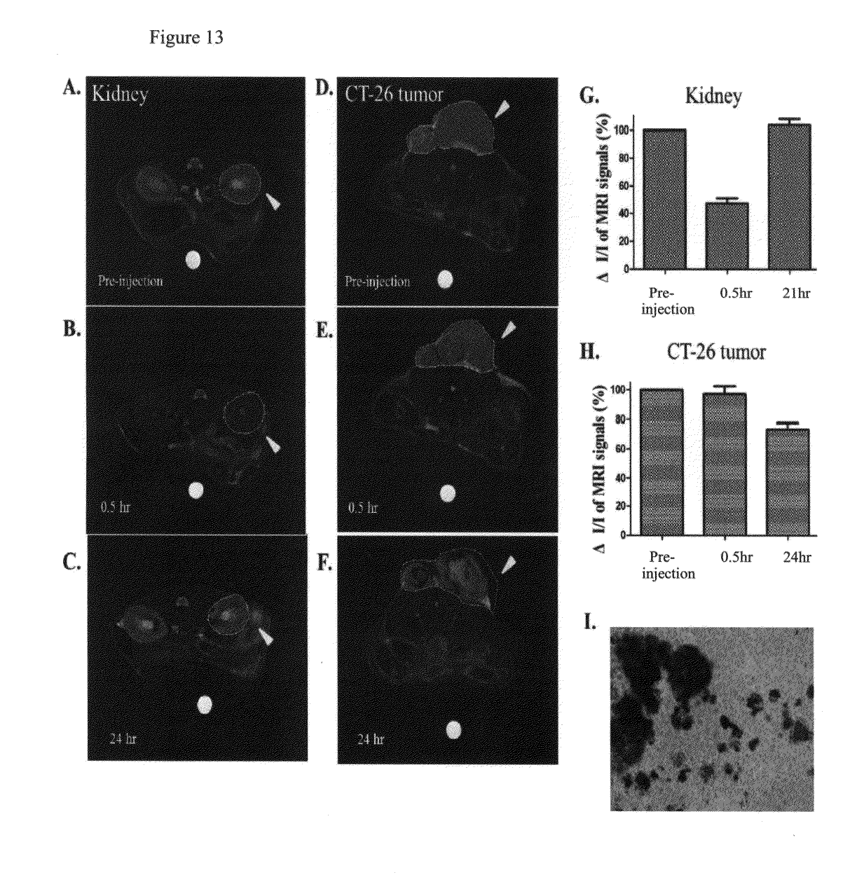

MNP-conjugated GBP was used as the targeted reagent for tumor imaging detection on MRI system. To monitor the targeting of MNP-GBP in colon carcinoma tumor bearing mouse, a 12-week-old female Balb/c mouse implanted CT-26 tumor on the back was injected intravenously with 150 μl MNP-GBP (0.06 emu/g) and MRI contrast data were collected within 24 h. MRI signal intensity of excretory kidney and CT-26 solid tumor bearing site were first detected before injection and set as 100% individually (FIGS. 13A and 13D). After MNP-GBP injection, MRI contrast of kidney dropped 55% within 0.5 h (FIG. 13B) and restored to 100% at 24 h (FIG. 13C). Yet, no difference in signal intensity was observed 0.5 hour after injection of MNP-GBP (FIG. 13E). MNP-GBP binding to the tumor, however, was evidently observed at 24 h after injection and MRI contrast intensity of CT-26 tumor tissue dropped about 30% as compared with pre-injection ones (FIG. 13F). MRI contrast intensity of kidney and tumor was thus quantified in FIGS. 13G and 13H, respectively. Tumor histology sections were stained with Prussian blue to detect iron nanoparticles within tumors 24 h after injection. As expected, MNP-GBP signal was not detected in normal tissue but appeared to recognize the edge of tumor tissues by Prussian blue signal at 24 h after injection (FIG. 11I). Importantly, the tumor histology sections at higher magnification suggested that MNP-GBP selectively targeted to CT-26 tumor tissue. These results suggested that GBP guided localization of MNP to tumor cells, strongly supporting the hypothesis that GBP not only bound cancer cells in vitro but also targeted tumor cells in vivo.

One skilled in the art readily appreciates that the present invention is well adapted to carry out the objects and obtain the ends and advantages mentioned, as well as those inherent therein. The kit, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of delivering an agent to a cancer cell in a mammalian subject, comprising:
(a) obtaining a peptide that binds to the cancer cell, wherein the peptide comprises a cancer binding motif having the amino acid sequence of SEQ ID NO: 1, wherein the peptide is conjugated or fused to the agent for targeting the cancer cell, wherein the conjugation or fusion is at N-terminus, C-terminus or both termini of the peptide; and
(b) exposing the peptide to a population of cells suspected of containing the cancer cell.

2. (canceled)

3. The method of claim 1, wherein the mammalian subject is a human subject.

4. The method of claim 1, wherein the population of cells is selected from the group consisting of blood and circulating tumor cells.

5. The method of claim 1, wherein the cancer is epithelial cancer.

6. The method of claim 5, wherein the epithelial cancer is adenocarcinoma or squamous cell carcinoma.

7. The method of claim 6, wherein the adenocarcinoma is high metastasis adenocarcinoma.

8. The method of claim 6, wherein the adenocarcinoma is selected from lung adenocarcinoma and colon adenocarcinoma.

9. The method of claim 1, further comprising detecting cancer cells in said population.

10. The method of claim 1, further comprising diagnosing cancer.

11. The method of claim 1, wherein the agent is a therapeutic agent or an imaging agent.

12. The method of claim 11, wherein the agent and peptide are administered to an individual having or suspected of having cancer to treat or image.

13. The method of claim 11, wherein the therapeutic agent is a drug, a chemotherapeutic agent, a radioisotope, a pro-apoptosis agent, an anti-angiogenic agent, a survival factor, an anti-apoptotic agent, an enzyme, a hormone, a hormone antagonist, a cytokine, a cytotoxic agent, a cytocidal agent, a cytostatic agent, a growth factor, a peptide, a protein, an antibiotic, an antibody, a Fab fragment of an antibody, a hormone antagonist, a nucleic acid, an antigen, a virus, a bacteriophage, a bacterium, a liposome, a microparticle, a magnetic bead, a microdevice, a yeast cell, a mammalian cell, a cell or an expression vector.

14. The method of claim 13, wherein the agent is an anti-angiogenic agent selected from the group consisting of thrombospondin, angiostatin5, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-B, thrombospondin, 2-methoxyoestradiol, proliferin-related protein, carboxiamidotriazole, CM 101, Marimastat, pentosan polysuiphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, Docetaxel, polyamines, a proteasome inhibitor, a kinase inhibitor, a signaling peptide, accutin, cidofovir, vincristine, bleomycin, AGM-1470, platelet factor 4 and minocycline.

15. The method of claim 13, wherein the agent is a cytokine selected from the group consisting of interleukin 1 (IL-1), IL-2, IL-5, IL-10, IL-11, IL-12, IL-18, interferon-γ (IF-γ), IF-α, IF-β, tumor necrosis factor-α (TNF-γ), or GM-CSF (granulocyte macrophage colony stimulating factor).

16. The method of claim 11, wherein the imaging agent is a tracing cargo selected from the group consisting of fluorescence tag, chemiluminescence protein, radioisotope, and magnetic nanoparticle.

17. The method of claim 16, wherein the imaging agent is a radioisotope selected from the group consisting of astatine-211, carbon-14, chromium-51, chlorine-36, cobalt-57, cobalt-58, copper-67, Eu-152, gallium-67, hydrogen-3, iodine-123, iodine-125, iodine-131, indium-111, iron-59, phosphorus-32, rhenium-186, rhenium-188, selenium-75, sulphur-35, technicium-99m and yttrium-90.

18. (canceled)

19. A kit for applying the method of claim 1, comprising a peptide that binds to a cancer cell in a mammalian subject, wherein the peptide comprises a cancer binding motif having the amino acid sequence of SEQ ID NO: 1.

20. (canceled)
